(19) United States
(12) Patent Application Publication
(10) Pub. No.: US 2015/0083135 A1
(43) Pub. Date: Mar. 26, 2015

(54) VENTILATOR TURBINE-BASED VOLUME-CONTROLLED VENTILATION METHOD

(71) Applicant: Beijing Aeonmed Co., Ltd., Beijing (CN)

(72) Inventor: Jie Cheng, Beijing (CN)

(21) Appl. No.: 14/396,311

(22) PCT Filed: Oct. 22, 2013
(86) PCT No.: PCT/CN2013/085724

§ 371 Date: Oct. 22, 2014

(30) Foreign Application Priority Data
Dec. 26, 2012 (CN) 201210576678.5

(51) Int. Cl. A61M 16/00; A61M 16/20; A61M 16/08; A61M 16/10

(52) U.S. Cl. CPC A61M 16/0069; A61M 16/0003; A61M 16/1005; A61M 16/204; A61M 16/205; A61M 16/0883; A61M 2016/0027; A61M 2016/003; A61M 2016/103; A61M 2205/3334
USPC 128/204.23

(57) ABSTRACT

A ventilator turbine volume-controlled ventilation method comprises the main steps of: the ventilator is started up, a control unit in the ventilator issues a rotation speed U control instruction to a turbine driver, the turbine driver drives a turbine motor, and then the control unit detects the breathing state of a patient, if the patient needs to inhale air, proceeds to an inhalation phase control, and, if the patient needs to exhale air, proceeds to an exhalation phase control, where the inhalation phase control is implemented by the control unit that outputs driving voltage V1 to regulate the extent to which an inhalation valve is opened, and the exhalation phase control is implemented by the control unit that outputs driving voltage V2 to regulate the extent to which an exhalation valve is opened.

### TECHNICAL FIELD

The present application relates to a volume-controlled ventilation field, in particular, to a ventilator turbine-based volume-controlled ventilation method.

### BACKGROUND

Volume-controlled ventilation (VCV) is a basic ventilation mode commonly used in a ventilator. A controlling process of the VCV is as follows: a pneumatic device generates inspiratory positive pressure to press air to a lung of a patient, and then the air is exhaled by constriction of the lung, further, the pneumatic device carries out respiratory ventilation according to preset parameters such as a frequency, a tidal volume, a respiratory quotient, and an oxygen concentration if the patient cannot breathe autonomously, and detects an autonomous breathing ability of the patient and carries out ventilation synchronously with the patient if the patient can breathe autonomously.

Nowadays, in the controlling process of the VCV, the air source pressure is typically supplied by an air compressor or other air source equipment, so that the ventilator is required to be near to the equipment supplying the air source pressure, thereby substantially limiting a moving range of the ventilator. Further, such a way of supplying pressure by the air source cannot satisfy the requirement for using the ventilator in a wild environment.

A turbine can provide an air source in various environments such as the wild environment. During controlling the turbine, however, considering that rotating fluid is unsteady, when the turbine rotates in a low speed, a flow of the fluid cannot meet the actual needs and thus problems such as an excessively low flow rate are likely caused; and when the turbine rotates in a high speed, a waveform of the flow of the fluid is likely to jitter, and thus it is difficult to achieve a controlled constant flow. Therefore, the turbine is generally not considered to provide the air source for the ventilator.

### SUMMARY

The known volume-controlled ventilation method is disadvantageous in that it is difficult to achieve constant flow control, real time control, and synchronous control when a turbine is used in a ventilator. In order to overcome the disadvantages, a technical problem to be solved by the present disclosure is to provide a ventilator turbine-based volume-controlled ventilation method which can achieve constant flow control and synchronous and realtime control on the turbine by combining a part of operation parameters of the ventilator with rotation speed control of the turbine, hence the turbine can provide an air source to the ventilator in various environments without the air source, such as a wild environment. Additionally, an inspiratory valve of the ventilator is controlled by PID to shorten a response time taken for the flow rate to reach a steady state, so as to meet the actual respiratory situation of the patient.

The technical solutions below are employed to achieve the above objects.

A ventilator turbine-based volume-controlled ventilation method, includes:

a step S00 of starting a ventilator, where a control unit in the ventilator sends a control instruction for controlling a rotation speed U to a turbine driver so that the turbine driver drives a turbine connected with the turbine driver;

a step S10 of detecting respiration status of a patient by the control unit, where the step S20 is performed if the patient needs inspiration and the step S30 is performed if the patient needs expiration;

a step S20 of outputting a driving voltage V₁, by the control unit via inspiratory phase control, to adjust an opening degree of an inspiratory valve, where the step S30 is performed after the inspiratory phase control is finished;

a step S30 of outputting a driving voltage V₂, by the control unit via expiratory phase control, to adjust an opening degree of an expiratory valve, where the step S20 is performed after the expiratory phase control is finished; and

a step S40 of turning off the ventilator and stopping supplying air to the patient.

Further, the step S40 is performed if the supplying air to the patient needs to be stop in the step S20 and the step S30.

Further, in the step S20, the control unit detects a monitored pressure value of a breathing circuit by a pressure sensor connected with the control unit in real time, and the inspiratory phase control is finished and the step S30 is performed if the monitored pressure value is larger than an alarm value or an inspiratory time is over.

Further, in the step S30, the control unit samples an airway pressure value of the patient by a pressure sensor connected with the control unit in real time, and the expiratory phase control is finished and the step S20 is performed if the airway pressure value is less than a difference value between a positive end-expiratory pressure Peep and a triggering pressure value or an expiratory time is over.

Further, in the step S00, the rotation speed U of the turbine is computed by a formula of:

U=R_{—}VCV*Qtarget+Ti*Qtarget/C_{—}VCV+PEEP_{—}Set,

where R_VCV represents system resistance, Qtarget represents a preset flow rate, Ti represents an inspiratory time, C_VCV represents system compliance, and PEEP_Set represents a preset positive end-expiratory pressure value.

Further, the preset flow rate Qtarget is computed by a formula of:

Qtarget=TV/T,

where TV represents a preset value of a tidal volume, and T represents an inspiratory time.

Further, in the driving voltage V₁ in the inspiratory phase control is computed by formulas of:

$$feedforward\_Ctrl = \frac{TV}{T} * ( T\_now / lp\_C + lp\_R ) * K ; and$$

$${V}_{1} = kp\_F * ( \frac{TV}{T} - lp\_F ) + feedforward\_Ctrl .$$

Where, TV represents a preset value of the tidal volume, T represents an inspiratory time, K₁ represents a proportional coefficient, T_now represents a real time, lp_C represents a post-filtering lung compliance, lp_R represents post-filtering lung airway resistance, feedforward_Ctrl represents a feedforward value, kp_F represents a testing proportional coefficient, and lp_F represents a feed backward value.

Further, the proportional coefficient K₁ is a slope of a flow-voltage curve of an inspiratory valve.

Further, in the driving voltage V₂ in the expiratory phase control is computed by a formula of:

V₂=K₂*(Peep+DP)+B,

where Peep represents positive end-expiratory pressure, DP represents a difference value between a preset positive end-expiratory pressure value and a monitored positive end-expiratory pressure value, K₂ represents a coefficient, and B represents a coefficient.

Further, the coefficient K₁ and the coefficient B are two parameters of an equation of an air pressure-voltage curve of the expiratory valve, wherein the coefficient K₁ is a slope of the equation and the coefficient B is intercept of the equation.

The beneficial effects of the present disclosure are that: the method achieve constant flow control and synchronous and realtime control on the turbine by combining a part of operation parameters of the ventilator such as system resistance R_VCV, system compliance C_VCV, and a preset positive end-expiratory pressure value PEEP_Set with rotation speed control of the turbine, so that the ventilator is applicable to environments without air source such as a wild environment. Additionally, a PID control manner is employed to control an inspiratory valve of the ventilator in an inspiratory phase, so as to effectively shorten a response time taken for the flow rate to reach a steady state.

### DESCRIPTION OF DRAWINGS

FIG. 1 is a flowchart of a ventilator turbine-based volume-controlled ventilation method according to an embodiment of the disclosure;

FIG. 2 is a flowchart of inspiratory control of the ventilator turbine-based volume-controlled ventilation method according to the embodiment of the disclosure; and

FIG. 3 is a flowchart of expiratory control of the ventilator turbine-based volume-controlled ventilation method according to the embodiment of the disclosure.

### DETAILED DESCRIPTION OF THE EMBODIMENTS

The technical solutions of the present disclosure are described in more detail below in combination with accompanying drawings and embodiments.

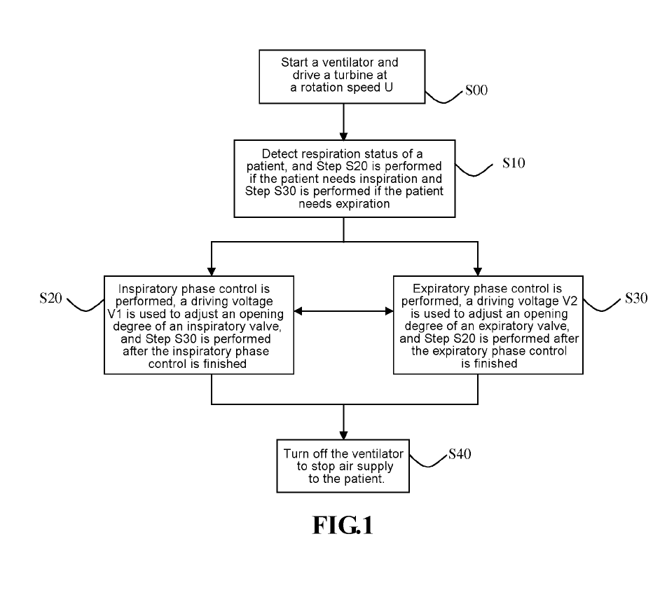

FIG. 1 is a flowchart of a ventilator turbine-based volume-controlled ventilation method according to an embodiment of the disclosure.

The ventilator turbine-based volume-controlled ventilation method is implemented according to Steps S00 to S40 as below.

At Step S00, a ventilator is started. A control unit in the ventilator sends a control instruction for controlling a rotation speed U to a turbine driver, so that the turbine driver drives a turbine connected with the turbine driver. The rotation speed U of the turbine is computed by a formula as follow:

U=R_{—}VCV*Qtarget+Ti*Qtarget/C_VCV+PEEP_Set,

where, R_VCV represents system resistance, Qtarget represents a preset flow rate, Ti represents an inspiratory time, C_VCV represents system compliance, and PEEP_Set represents a preset positive end-expiratory pressure (PEEP) value.

The system resistance R_VCV and the system compliance C_VCV are determined by system design parameters of the ventilator. The preset PEEP value PEEP_Set is determined by situations of the individual patient.

The preset flow rate Qtarget is computed by a formula of Qtarget=TV/T .

TV represents a preset value of a tidal volume, and T represents an inspiratory time.

The preset value TV of the tidal volume is computed according to an ideal weight of the patient, and the inspiratory time T is synchronous with the inspiratory time of the patient.

The constant-flow control and the synchronous realtime control of the turbine can be achieved by combing the operation parameters of the ventilator such as the system resistance R_VCV , the system compliance C_VCV and the preset PEEP value PEEP_Set with the rotation speed control of the turbine. Therefore, the turbine can be employed to provide the air source for the ventilator so that the ventilator can be used in various environments without air source, like in a wild environment.

At Step S10, respiration status of the patient is detected by the control unit, and Step S20 is performed if the patient needs inspiration and Step S30 is performed if the patient needs expiration. The respiration status of the patient can be detected by various ways, such as by a way of detecting a carbon dioxide concentration change by a carbon dioxide sensor during the breath process, or by a way of detecting a concentration curve of end-expiratory carbon dioxide.

Step S20 includes control of an inspiratory phase. The control of the inspiratory phase refers to a whole process that the control unit outputs a driving voltage V₁ to adjust an opening degree of an inspiratory valve. In the control of the inspiratory phase, the driving voltage V₁ is computed by formulas as follows:

$$\begin{matrix}feedforward\_Ctrl = \frac{TV}{T} * ( T\_now / lp\_C + lp\_R ) * K , & 1 \\ {V}_{1} = kp\_F * ( \frac{TV}{T} - lp\_F ) + feedforward\_Ctrl . & 2\end{matrix}$$

Where, TV represents a preset value of the tidal volume, T represents an inspiratory time, K₁ represents a proportional coefficient, T_now represents a real time, lp_C represents a post-filtering lung compliance, lp_R represents post-filtering lung airway resistance, feedforward_Ctrl represents a feedforward value, kp_F represents a testing proportional coefficient, and lp_F represents a feed backward value.

The post-filtering lung compliance lp_C and the post-filtering lung airway resistance lp_R can be calculated from post-filtering values detected by a respiratory flow monitor and a pressure sensor sampling probe, the feedforward value feedforward_Ctrl is a voltage value, the feed backward value lp_F is a flow value detected by a flow sensor, and the testing proportional coefficient kp_F is a proportional coefficient for proportional-integral-derivative (PID) control, which may be determined by testing a PID controller and determines the time taken to reach a target flow rate, where jitters will be caused if the proportional coefficient is too large, and the time taken to reach the target flow rate will be too long if the proportional coefficient is too small. The proportional coefficient K₁ is a slope of a flow-voltage curve of an inspiratory valve and is determined by testing the inspiratory valve.

The formula \textcircled{1} and formula \textcircled{2} form closed loop PID control with a feedforward signal and a feed backward signal. The closed loop PID control can shorten a response time taken for the flow rate to reach a steady state, namely, the target flow rate can be reached rapidly and effectively maintained steady.

Step S30 is performed after the control of the inspiratory phase is finished.

Step S30 includes control of an expiratory phase. The control of the expiratory phase refers to a whole process that the control unit outputs a driving voltage V₂ to adjust an opening degree of an expiratory valve. In the control of the expiratory phase, the driving voltage V₂ is computed by a formula as follows:

V₂=K_{2*}(Peep+DP)+B.

Where, Peep represents positive end-expiratory pressure, DP represents a difference value between a preset PEEP value and a monitored PEEP value, K₂ represents a coefficient, and B represents a coefficient.

The positive end-expiratory pressure Peep plus the difference value DP forms the closed loop control. If the value of Peep is too large in the previous cycle, DP is less than zero; and if the value of Peep is too small in the previous cycle, DP is larger than zero, thereby making the positive end-expiratory pressure peep more steady, so that the gas flow is exhaled more steadily in the expiratory process to meet the actual expiratory status of the patient.

Additionally, because the expiratory valve is a linear proportional valve, an air pressure-voltage curve of the expiratory valve is approximately a straight line. Two parameters of a slope intercept form of the straight line respectively are the coefficients K₁ and B, where the coefficient K₁ is the slope of the straight line and the coefficient B is intercept of the straight line.

Step S20 is performed after the control of the expiratory phase is finished.

To stop supplying the air to the patient in Steps S20 and S30, Step S40 is performed.

At Step S40, the ventilator is turned off and supplying air to the patient is stopped.

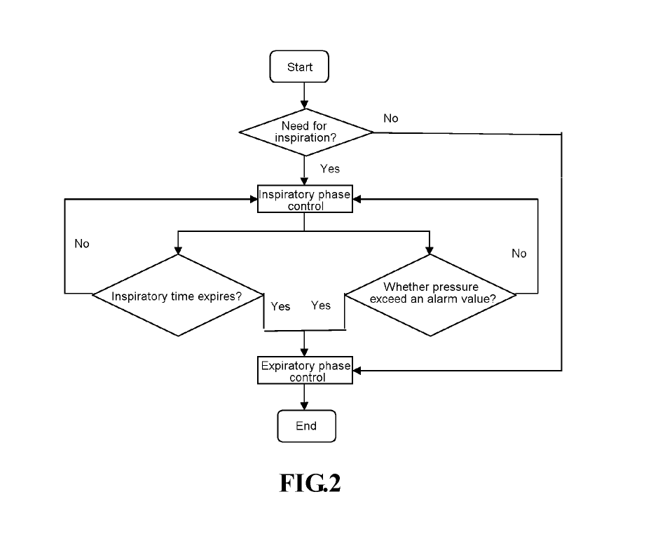

FIG. 2 is a flowchart of inspiratory control of the ventilator turbine-based volume-controlled ventilation method according to the embodiment of the disclosure.

At Step S10, the respiration status of the patient is detected by the control unit, and the control of the inspiratory phase, namely Step S20, is carried out if the patient needs inspiration. Meanwhile the control unit detects a monitored pressure value of a breathing circuit by a pressure sensor connected with the control unit in real time. The control of the inspiratory phase is finished and the control of the expiratory phase (namely Step S30) is performed if the monitored pressure value is larger than an alarm value or the entire inspiratory process is finished, namely the inspiratory time is over.

Additionally, Step S40 is performed if it is required to stop supplying air to the patient.

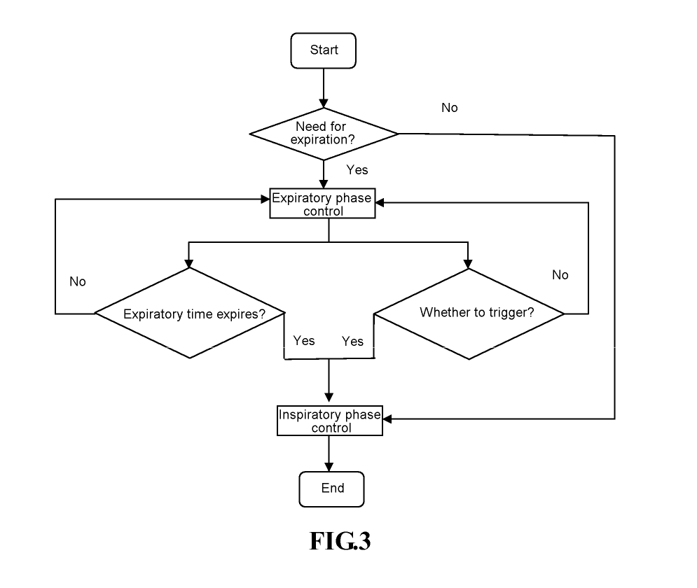

FIG. 3 is a flowchart of expiratory control of the ventilator turbine-based volume-controlled ventilation method according to the embodiment of the disclosure.

At Step S10, the respiration status of the patient is detected by the control unit, and the control of the expiratory phase, namely Step S30, is performed if the patient needs expiration. The control unit samples an airway pressure value of the patient by a pressure sensor connected with the control unit in real time. The control of the expiratory phase is finished and the control of the inspiratory phase (namely Step S20) is performed if the airway pressure value is less than a difference value between the positive end-expiratory pressure Peep and a triggering pressure value or the entire expiratory process is finished, namely the expiratory time is over. Herein the triggering pressure value is a preset value that is preset according to the respiration situation of the patient and generally within a range of 3-20 cmH2O. For example, in the case that the triggering pressure value is preset as 3 cmH2O, if the value of the end-expiratory pressure Peep is 5 cmH2O, when the sampled airway pressure value is less than 5-3=2 cmH2O, it is determined that the patient wants to inhale, namely the inspiratory triggering condition is reached and the control of the inspiratory phase is performed.

Additionally, Step S40 is performed if it is required to stop supplying air to the patient.

The present disclosure is disclosed by preferred embodiment. The person skilled in the art should know that various corresponding changes and modifications of the present disclosure can be made without departing from the spirits and essences of the present disclosure. The present invention is not limited by the embodiments disclosed herein and other embodiments within the scope of the claims of the present disclosure should fall into the scope of protection of the present disclosure.

1. A ventilator turbine-based volume-controlled ventilation method, wherein a ventilator is provided with a turbine configured for providing an air source for the ventilator, and the ventilator turbine-based volume-controlled ventilation method comprises:
a step S00 of starting the ventilator, wherein a control unit in the ventilator sends a control instruction for controlling a rotation speed U to a turbine driver so that the turbine driver drives the turbine connected with the turbine driver;
a step S10 of detecting respiration status of a patient by the control unit, wherein the step S20 is performed if the patient needs inspiration and the step S30 is performed if the patient needs expiration;
a step S20 of outputting a driving voltage V₁, by the control unit via inspiratory phase control, to adjust an opening degree of an inspiratory valve, wherein the step S30 is performed after the inspiratory phase control is finished;
a step S30 of outputting a driving voltage V₂, by the control unit via expiratory phase control, to adjust an opening degree of an expiratory valve, wherein the step S20 is performed after the expiratory phase control is finished; and
a step S40 of turning off the ventilator and stopping supplying air to the patient.

2. The ventilator turbine-based volume-controlled ventilation method of claim 1, wherein the step S40 is performed if the supplying air to the patient needs to be stop in the step S20 and the step S30.

3. The ventilator turbine-based volume-controlled ventilation method of claim 1, wherein in the step S20, the control unit detects a monitored pressure value of a breathing circuit by a pressure sensor connected with the control unit in real time, and the inspiratory phase control is finished and the step S30 is performed if the monitored pressure value is larger than an alarm value or an inspiratory time is over.

4. The ventilator turbine-based volume-controlled ventilation method of claim 1, wherein in the step S30, the control unit samples an airway pressure value of the patient by a pressure sensor connected with the control unit in real time, and the expiratory phase control is finished and the step S20 is performed if the airway pressure value is less than a difference value between a positive end-expiratory pressure Peep and a triggering pressure value or an expiratory time is over.

5. The ventilator turbine-based volume-controlled ventilation method of claim 1, wherein in the step S00, the rotation speed U of the turbine is computed by a formula of:
U=R_{—}VCV*Qtarget+Ti*Qtarget/C_{—}VCV+PEEP_Set,
wherein R_VCV represents system resistance, Qtarget represents a preset flow rate, Ti represents an inspiratory time, C_VCV represents system compliance, and PEEP_Set represents a preset positive end-expiratory pressure value.

6. The ventilator turbine-based volume-controlled ventilation method of claim 5, wherein the preset flow rate Qtarget is computed by a formula of Qtarget=TV/T, wherein TV represents a preset value of a tidal volume, and T represents an inspiratory time.

7. The ventilator turbine-based volume-controlled ventilation method of claim 1, wherein the driving voltage V₁ in the inspiratory phase control is computed by formulas of:
$$feedforward\_Ctrl = \frac{TV}{T} * ( T\_now / lp\_C + lp\_R ) * K ; and$$
$${V}_{1} = kp\_F * ( \frac{TV}{T} - lp\_F ) + feedforward\_Ctrl ,$$
wherein TV represents a preset value of the tidal volume, T represents an inspiratory time, K represents a proportional coefficient, T_now represents a real time, lp_C represents a post-filtering lung compliance, lp_R represents post-filtering lung airway resistance, feedforward_Ctrl represents a feedforward value, kp_F represents a testing proportional coefficient, and lp_F represents a feed backward value.

8. The ventilator turbine-based volume-controlled ventilation method of claim 7, wherein the proportional coefficient K is a slope of a flow-voltage curve of an inspiratory valve.

9. The ventilator turbine-based volume-controlled ventilation method of claim 1, wherein the driving voltage V₂ in the expiratory phase control is computed by a formula of:
V₂=K₂*(Peep+DP)+B,
wherein Peep represents positive end-expiratory pressure, DP represents a difference value between a preset positive end-expiratory pressure value and a monitored positive end-expiratory pressure value, K₂ represents a coefficient, and B represents a coefficient.

10. The ventilator turbine-based volume-controlled ventilation method of claim 9, wherein the coefficient K₂ and the coefficient B are two parameters of an equation of an air pressure-voltage curve of the expiratory valve, wherein the coefficient K₂ is a slope of the equation and the coefficient B is intercept of the equation.
